(19) United States
(12) Patent Application Publication
(10) Pub. No.: US 2022/0395227 A1
(43) Pub. Date: Dec. 15, 2022

(54) ALIGNMENT TOOL FOR CHEST-WORN SENSOR DEVICE

(71) Applicant: Analog Devices, Inc., Wilmington, MA (US)

(72) Inventors: David BOLOGNIA, Charlestown, MA (US); Jiawen BAI, Brighton, MA (US)

(73) Assignee: Analog Devices, Inc., Wilmington, MA (US)

(21) Appl. No.: 17/344,780

(22) Filed: Jun. 10, 2021

(51) Int. Cl. A61B 5/00; A61B 5/0531; A61B 5/28

(52) U.S. Cl. CPC A61B 5/684; A61B 5/6831; A61B 5/0531; A61B 5/4875; A61B 5/6823; A61B 5/28; A61B 2560/04; A61B 2562/0219; A61B 2562/0271

(57) ABSTRACT

An alignment tool helps a patient position a chest-worn sensor device, such as a device for daily at-home monitoring of cardiopulmonary health conditions. The alignment tool includes a sensor connector that engages with the sensor device and a strap. The strap includes a first portion that has a protruding region sized to fit into the patient's suprasternal notch. The strap includes a second portion that engages with the sensor connector. The sensor connector and the first portion of the strap with the suprasternal notch protrusion are separated by a configurable length. A healthcare provider may set the length based on the distance between the patient's suprasternal notch and heart.

### FIELD OF THE DISCLOSURE

The present application relates generally to systems, apparatus, and methods of aligning a chest-worn sensor for managing medical or health conditions in human subjects.

### BACKGROUND

Congestive heart failure (CHF) is a known cardiac condition in which damaged heart muscle loses its ability to pump sufficient amounts of blood to meet the body's demands. In the early stages of CHF, such an inability to pump sufficient amounts of blood may occur only while a human exercises. However, in more advanced stages of CHF, an inability to pump sufficient amounts of blood may occur even while the human subject is at rest. CHF is one of the most commonly diagnosed cardiac conditions in hospital patients over the age of 65, and one of the most frequent reasons for such patients' readmission to hospitals in a time duration of 30 days. In recent years, 30-day hospital readmission expenses for CHF have increased to $1.8 billion per year, with approximately $13,000 being allotted for each readmission at a 25% readmission rate. Some of the reasons for such patients' readmission to hospitals can include, but are not limited to, (1) patient non-compliance with regard to diet and medication, which can result in excess fluid in the lungs or extreme dehydration, (2) incomplete titration of medication dosages, which often need to be modified as a patient moves from the hospital environment back to his or her home, and (3) atrial fibrillation, which can onset after the patient's discharge from the hospital.

Management of CHF in patients following discharge from the hospital has traditionally focused on monitoring the patients' fluid retention using sensors incorporated in implantable cardiac devices, such as implantable cardioverter defibrillators (ICDs), cardiac resynchronization therapy-defibrillators (CRT-Ds), or pacemakers. Such implantable cardiac devices can detect developing pulmonary congestion in a patient by measuring the patient's thoracic fluid impedance. For example, an implantable cardiac device such as an ICD, CRT-D, or pacemaker can be configured to pass an electrical current across a patient's lung, and to measure the resulting intra-thoracic impedance. As the patient's thoracic fluid accumulates during pulmonary congestion, conductance across the patient's lung increases, causing a corresponding decrease in impedance indicative of the level of thoracic fluid accumulation. Such implantable cardiac devices can also be interrogated by hospital clinicians, allowing the hospital clinicians to monitor the patient's fluid status and to receive early warnings of changes that may signal an impending fluid overload. Based on the patient's monitored fluid status, the hospital clinicians may then determine whether or not it would be appropriate to readmit the patient to the hospital for further monitoring and/or treatment.

Convention implantable cardiac devices have several disadvantages. For example, conventional implantable cardiac devices typically include one or more sensors configured to provide a single or limited number of sensing modalities, such as a modality for detecting a human subject's fluid retention. However, monitoring and/or tracking the CHF status of a human subject based on just a single or limited number of sensing modalities can often lead to false positives, resulting in unnecessary hospital readmissions that can increase healthcare costs. In addition, the implantable nature of such conventional cardiac devices can increase surgical risks, as well as the incidence of infection. Furthermore, the implantable nature of such conventional cardiac devices limits the availability of the devices to patients, since only patients qualified for the surgery to insert the implant can receive the device.

To overcome these disadvantages, external and non-invasive devices for at-home detection and monitoring of CHF and/or other health conditions, such as chronic obstructive pulmonary disease (COPD), are being developed. External devices can provide multiple sensing modalities that non-invasively gather data, and can at least partially analyze, trend, and/or reduce data from the various sensing modes. For example, an external device may include one or more electrodes, heart sounds sensors, ultrasound sensors, and photoplethysmography sensors. A multi-modality sensing device increases the positive detection of potentially problematic CHF conditions while decreasing false positives, which can reduce the number of unnecessary hospital readmissions, shorten hospital stays, and reduce hospital costs. Moreover, an external device that can be conveniently employed by a patient following discharge from the hospital allows the patient as well as hospital clinicians to monitor the subject's CHF and/or COPD status without the risks or surgery and infection.

### BRIEF DESCRIPTION OF THE DRAWINGS

FIG. 1 illustrates a sensor device positioned on a chest of a wearer, according to some embodiments of the present disclosure;

FIG. 2 illustrates an alignment tool positioning the sensor device on the chest of the wearer, according to some embodiments of the present disclosure;

FIG. 3 illustrates a front view of an example alignment tool, according to some embodiments of the present disclosure;

FIG. 4 illustrates a front view of the alignment tool with the UI housing removed, according to some embodiments of the present disclosure;

FIG. 5 illustrates a perspective view of the example alignment tool, according to some embodiments of the present disclosure;

FIG. 6 illustrates a perspective view of a second example alignment tool, according to some embodiments of the present disclosure; and

FIG. 7 illustrates a top view of a third example alignment tool, according to some embodiments of the present disclosure.

### DESCRIPTION OF EXAMPLE EMBODIMENTS OF THE DISCLOSURE

### Overview

The systems, methods and devices of this disclosure each have several innovative aspects, no single one of which is solely responsible for all of the desirable attributes disclosed herein. Details of one or more implementations of the subject matter described in this specification are set forth in the description below and the accompanying drawings.

As described above, external devices for at-home monitoring of CHF, COPD, or other health conditions provide several advantages for patients and clinicians, including eliminating the need for surgical implementation which increases risks and costs to the patient, and collecting a broader set of data with multiple sensing modalities. However, a patient using an external, removable device at home must be able to properly position the device so that the device collects usable data. For example, an external device may include two or more electrodes for measuring thoracic impedance across the wearer's chest and an acoustic sensor to measure the wearer's heart sound (e.g., S3 or S4 heart sounds). In some embodiments, e.g., for COPD detection, the acoustic sensor, or one or more additional acoustic sensors, may capture lung sounds. In some embodiments, the electrodes, or one or more additional electrodes, may be used to collect electrocardiogram (ECG) signals. If the electrodes and/or other sensors are not properly positioned on the wearer's chest, the device may not capture the intended signals. For example, improperly positioned electrodes may not measure impedance across the expected portion or portions of the wearer's chest, or misplaced acoustic devices may capture attenuated heart sounds and/or lung sounds, or even fail to capture the target sounds.

The alignment tool described herein enables patients to correctly position an external sensor device across their chests at home for routine monitoring. For example, a doctor may prescribe a device to a patient and request that the patient use the device to obtain measurements twice per day, e.g., at the morning and at night. At each measurement session, the patient can use the alignment tool to properly position the device on his or her chest. After the device has completed its measurement sequence, the patient removes the device until the next use. With repeated use, the patient may learn to position the device without the alignment tool, or the patient may use the alignment tool before each measurement session.

The alignment tool incudes a sensor connector to engage with a portion of the sensing device and a strap connected to the sensor connector. One end of the strap is sized to fit into the patient's suprasternal notch, and another portion of the strap engages with the sensor connector. The alignment tool indicates the distance below the patient's suprasternal notch that the sensor should be positioned. The strap is oriented vertically along the patient's body, so that the sensor is positioned directly below the patient's suprasternal notch at the distance indicated by the strap.

Due to variation in body shapes and sizes, the proper sensor position below the suprasternal notch varies from person to person. The alignment tool is initially configured by a doctor or other healthcare worker for a specific patient's body, e.g., the healthcare worker selects a length for the alignment tool strap based on the distance between the patient's suprasternal notch and the patient's heart. The healthcare worker then instructs a patient on how to use the alignment tool, e.g., how to find the patient's suprasternal notch and use the alignment tool to place the device at a particular location relative to the suprasternal notch. After this initial setup, the patient can use the alignment tool independently to position the alignment tool. If the patient is not able to independently position the device (e.g., due to mobility issues), a family member, home healthcare worker, or another person may use the alignment tool to place the device on the patient's body and assist the patient with the measurement procedure.

### Example Sensor Device

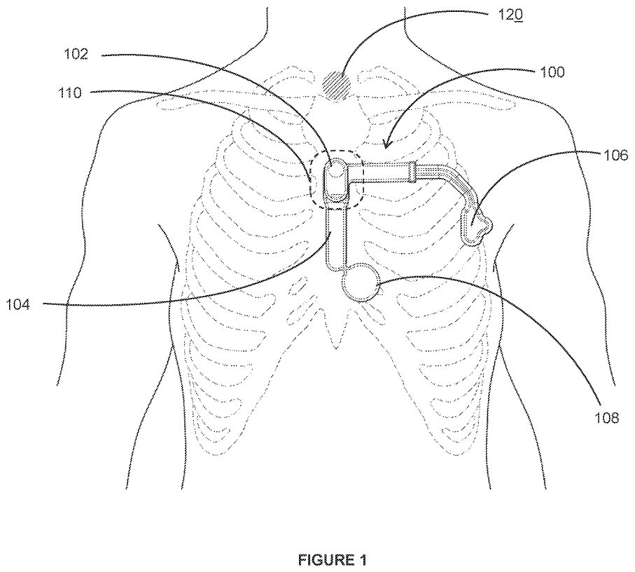

FIG. 1 illustrates a sensor device 100 positioned on a chest of a wearer, according to some embodiments of the present disclosure. The sensor device 100 includes a user interface (UI) housing 102 that is physically connected and electrically coupled to two islands 106 and 108. A base material 104, such as a rubber or other flexible material, connects the UI housing 102 to the two islands 106. One of the islands 106 is positioned under the left arm area, near the middle axillary line. The other island 108 is positioned just to the left of the sternum, e.g., at the left sternal border. The UI housing 102 and the islands 106 and 108 have an adhesive backing for adhering the sensor device 100 to the user's chest during a measurement procedure. A patient may wear the sensor device 100 during measurement sessions, e.g., for a period of several minutes, one or more times a day, and remove the sensor device 100 after each measurement session. The adhesive backing may be replaceable, e.g., once per week.

The sensor device 100 includes various sensors to capture data that may assist with management of one or more cardiopulmonary conditions, such as CHF or COPD. In one embodiment, the UI housing 102, which is positioned near the apex of the patient's heart, includes an electronic stethoscope or other acoustic sensor for capturing heart sounds. The islands 106 and 108 each include one or more electrodes, e.g., for capturing electrocardiogram (ECG) and/or bio-impedance measurements. In some embodiments, the sensor device 100 (e.g., the UI housing 102) includes additional sensors, such as a temperature sensor and an accelerometer to measure tilt (body position). The UI housing 102 further includes control circuitry for controlling the sensor device 100, e.g., to control the electrodes and other sensors, and to capture and store data. The UI housing 102 further includes one or more user interface features, such as a button for the patient to indicate when to start collecting measurements, and light, sound, or other outputs for providing information or alerts to the patient. The UI housing 102 may include a battery and one or more electronic interfaces, e.g., to connect to a base station to export measurement data, receive firmware updates, receive power, etc.

The sensors in the sensor device 100 capture a variety of data that can be used by a doctor to identify potential cardiopulmonary issues or other health concerns. The sensor device 100 or a connecting device, such as a base station, may transmit measurements from the sensor device 100 to a data portal (e.g., a web portal) available to the patient's doctor. This allows the doctor to remotely review the patient's measurements on a regular basis and, for example, determine whether to admit the patient to the hospital for further monitoring, or to change the patient's treatment plan. The sensor device 100 may capture, for example, skin temperature, thoracic impedance, change in thoracic impedance, respiration data, and tidal volume. The sensor device 100 (e.g., using the electrodes in the islands 106 and 108) also may capture an ECG and derive various metrics from the ECG, such as heart rate, length of the QRS complex, QT interval, and other metrics that may indicate cardiac abnormalities. In some embodiments, some of these metrics may be derived by another device that receives data from the sensor device 100, e.g., computation may be done by a base station that connects to the sensor device 100.

To accurately capture measurements indicative of the patient's cardiopulmonary health, the sensor device 100 must be properly positioned on the patient's chest. For example, if the UI housing 102 includes an electronic stethoscope, it can most accurately capture the heart sounds when positioned within a given range of the apex of the patient's heart. An example acceptable range 110 for the placement of the UI housing 102 is shown in FIG. 1; in FIG. 1, the UI housing 102 is placed in the center of this range, but the “correct” placement may include the range 110, e.g., of a centimeter or a few centimeters in the horizontal and/or vertical directions around the position of the UI housing 102 in FIG. 1. In other embodiments, the range may be smaller or greater, or the size of the range may differ between the horizontal and vertical directions (e.g., the range of acceptable positions may be greater in the vertical direction than in the horizontal direction).

The range 110 is centered along a center line of the patient's body, and may be defined as a distance in the vertical direction from the patient's suprasternal notch, shown as the shaded region 122. The suprasternal notch is a dip near the base of a person's neck, between the clavicles and above the manubrium of the sternum, and is also referred to as the jugular notch. The position of the UI housing 102 to the suprasternal notch 120 varies between different patient's based on their body shape and the relative positions of their suprasternal notch 120 and heart. For example, in some patient's, the UI housing 102 should be positioned a short distance (e.g., a few centimeters) below the suprasternal notch 120, and in other patients, the UI housing 102 should be positioned 10 centimeters or more below the suprasternal notch.

Proper positioning of the UI housing 102 can assist a patient in properly placing the islands 106 and 108. As noted above, the electrodes in the islands 106 and 108 are positioned on or near the middle axillary line and the left sternal border, respectively (e.g., within a few centimeters of these lines), to obtain a strong ECG signals and/or other measurements. The electrodes may also have target vertical positions relative to the patient's ribs, for example, the island 106 may be placed near the fifth intercostal space, and the island 108 may be placed near the fourth intercostal space.

### Example Alignment Tool

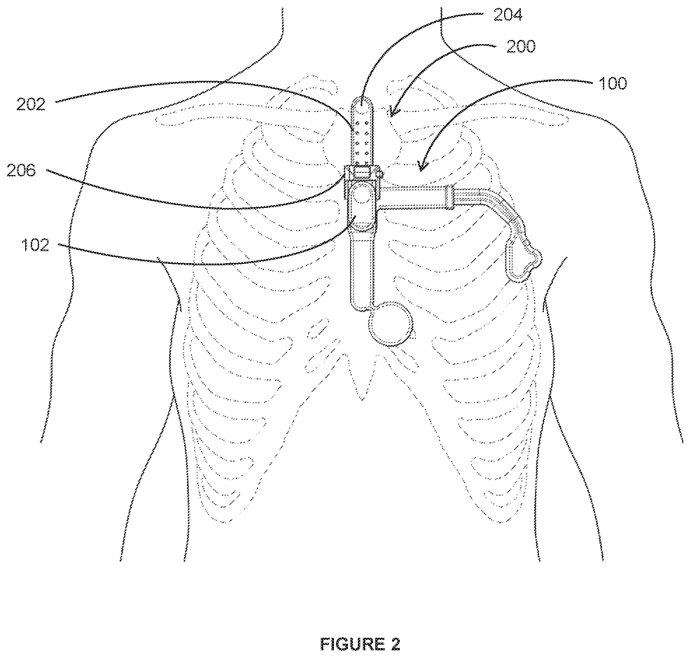

FIG. 2 illustrates an alignment tool 200 positioning the sensor device 100 on the chest of the wearer, according to some embodiments of the present disclosure. As described above, the position of the sensor device 100, and in particular, of the UI housing 102, can be described as a distance in the vertical direction from the patient's suprasternal notch 122. The suprasternal notch 122 is typically easy for a person to feel with their fingers. With basic instructions (e.g., when shown by a healthcare provider), patients can typically find their own suprasternal notch 122. The alignment tool 200 includes a strap 202 that includes a suprasternal notch indent 204 at one end that the patient inserts into their suprasternal notch. At the other end of the strap is a sensor connector 206 that engages with sensor device 100, e.g., by holding onto an upper portion of the UI housing 102. The strap 202 has a configurable length that sets the distance between the suprasternal notch and the UI housing 102. The length of the strap may be set by a doctor or other healthcare worker during an initial setup. When the patient places the suprasternal notch indent 204 into their suprasternal notch with the sensor connector 206 extending vertically downwards from the suprasternal notch indent 204, the UI housing 102 falls at the proper position, e.g., within the range 110 shown in FIG. 1. As noted above, the UI housing 102 may have an adhesive backing. Once the patient has positioned the UI housing 102 and adhered it to their chest, the patient may disengage the sensor connector 206 from the UI housing 102 and set aside the alignment tool 200. The patient then positions and adheres the islands 106 and 108 after the UI housing 102 has been positioned and adhered.

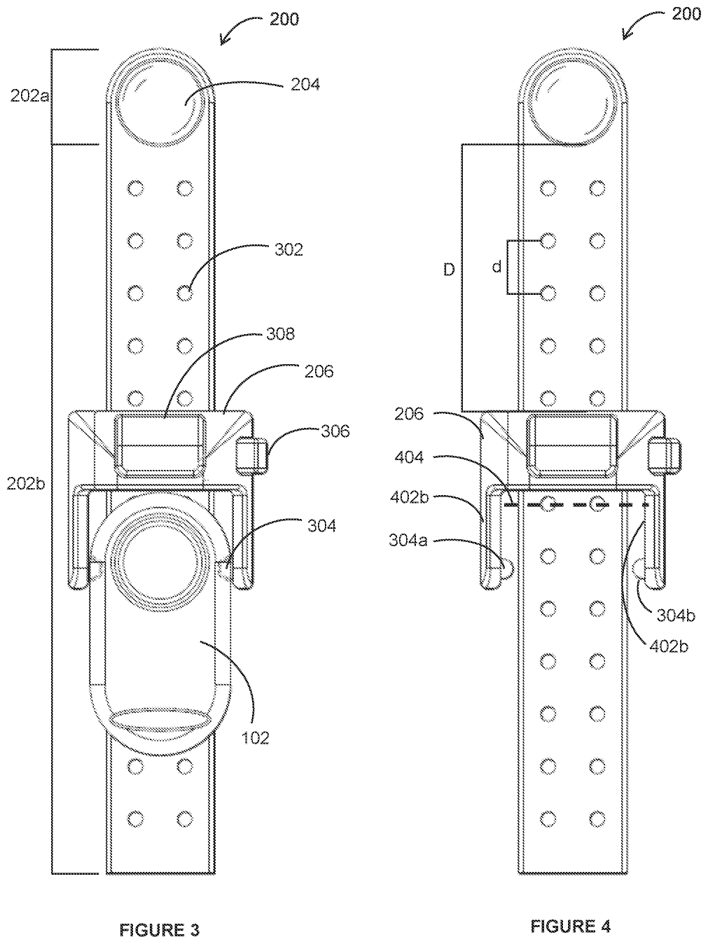

FIG. 3 illustrates a more detailed front view of the alignment tool 200, according to some embodiments of the present disclosure. The strap 202 has a first portion 202a that includes the suprasternal notch indent 204, and a second portion 202b that engages with the sensor connector 206. In the example configuration shown in FIG. 3, the second portion 202b of the strap extends through an opening in the sensor connector 206. The opening in the sensor connector 206 allows the second portion 202b of the strap to slide through sensor connector 206. An excess portion of the strap 202 sits behind the UI housing 102 in the view shown in FIG. 3. After the healthcare worker has selected the position for the sensor connector 206 along the strap 202, the healthcare worker may cut the excess portion of the strap 202 so that the adhesive back of the UI housing 102 is exposed, and the patient can adhere the UI housing 102 to their chest.

The suprasternal notch indent 204 is a protruding region that is sized to fit into a suprasternal notch of a patient. In the view shown in FIG. 3, the suprasternal notch indent 204 extends into the page, i.e., towards the back side of the alignment tool 200. In some embodiments, the suprasternal notch indent 204 includes a corresponding hollow portion on the front side of the strap 202 (the side shown in FIG. 3), i.e., on the opposite side of the strap 202 from the protruding region. This enables a patient to push a thumb or finger into the suprasternal notch indent 204. In other embodiments, the front side of the first portion of the strap 202a is smooth.

The second portion of the strap 202b is made of a material that does not permit stretching, such as a plastic or heavy cloth. The suprasternal notch indent 204 may be composed of the same material as the second portion of the strap 202b, or made from one or more different materials from the second portion 202b of the strap, e.g., the suprasternal notch indent 204 may include a rubber or silicone material, which may be more comfortable for the patient to place in their suprasternal notch 122. The strap 202 may be rigid or allow some bending, e.g., to conform to the shape of the patient's chest.

The sensor connector 206 engages with the UI housing 102 of the sensor device 100. For example, the sensor connector 206 includes a pair of insertable pins 304 that fit into and engage a corresponding pairs of receptacles (e.g., divots or blind holes) on the UI housing 102. The insertable pins 304 form a mechanical clasp that connect to the receptacles in the UI housing 102. The pins 304 may be composed of plastic, metal, or another rigid material. The sensor connector 206 includes a button 306 that, if pushed, causes one or both of the insertable pins 304 to retract to disengage the sensor connector 206 from the UI housing 102. In some embodiments, one of the insertable pins 304 is fixed (i.e., non-retracting), and pressing the button 306 to retract the other insertable pin enables the patient to remove the sensor connector 206 from the UI housing 102. In other embodiments, both of the pins 304 are retractable, and pressing the button 306 causes both pins 304 to retract. The design of the sensor connector 206 shown in FIG. 3 may enable a patient to position and adhere the UI housing 102 and then disengage and remove the sensor connector 206 with a single hand. At the same time, the patient may use their other hand to hold the suprasternal notch indent 204.

The portion of the strap 202 between the suprasternal notch indent 204 and the sensor connector 206 has a configurable length. In particular, the position of the sensor connector 206 along the second portion of the strap 202 sets a distance from the suprasternal notch indent 204 and the sensor connector 206. Because the suprasternal notch indent 204 is placed in the patient's suprasternal notch 122, and the relationship between the sensor connector 206 and the UI housing 102 is fixed, the configured length of the alignment tool 200 positions the UI housing 102 at the desired distance below the patient's suprasternal notch 122. A locking mechanism 308 is used to set the sensor connector 206 at the desired position along the second portion of the strap 202.

In the example shown in FIG. 3, the second portion of the strap 202b has a series of paired holes 302 running lengthwise along the strap 202. The locking mechanism 308 includes a set of corresponding pins that engage with one pair of the holes 302 to set the length. The locking mechanism 308 may be a button that pushes the pins through the selected pair of holes. The pins may be fixedly inserted into the selected holes, so that after initial configuration by a healthcare provider, the patient or another party cannot change the distance between the sensor connector 206 and the suprasternal notch indent 204. For example, the pins may engage with a pin retaining mechanism on the opposite side of the sensor connector 206 shown in FIG. 3. In other embodiments, the pins may be retractable and the length reconfigured, e.g., so the alignment tool 200 may be used by a different patient. While the strap 202 is shown as having pairs of holes 302, so that the locking mechanism 308 engages two holes to set the strap position, in other embodiments, the locking mechanism 308 may engage a single hole with a single pin or more than two holes with three or more pins, and the holes along the strap 202 are configured accordingly.

FIG. 4 illustrates a front view of the alignment tool 200 with the UI housing 102 removed, according to some embodiments of the present disclosure. FIG. 4 shows the two pins 304a and 304b that engage with the UI housing 102. As noted above, one or both of the pins 304 may retract, e.g., pin 304b moves rightward (in the orientation of FIG. 4) into the arm 402b of the sensor connector 206. In other embodiments, the sensor connector 206 may disengage from with the UI housing 102 in a different way. For example, one or both of the arms 402a and 402b may slide upwards (towards the suprasternal notch indent 204) or outwards (away from the UI housing 102) to disengage the UI housing 102. In some embodiments, the sensor connector 206 does not engage with the UI housing 102 using the pins 304 but through some other removeable connection mechanism, such as a hook-and-loop connection or a magnetic connection.

FIG. 4 also illustrates two distances on the strap 202. The larger distance D illustrates the configurable distance between the sensor connector 206 and the suprasternal notch indent 204. The smaller distance d illustrates a distance between consecutive holes 302 along the length of the strap 202. The distance d may be, for example, approximately 1 centimeter, e.g., in a range of 0.5 cm to 2 cm or any range therein, such as between 0.75 cm and 1.25 cm, or between 0.9 cm and 1.1 cm. The distance d may be selected based on the acceptable positioning range of the UI housing 102, e.g., with a larger distance d for a larger positioning range, or a smaller distance d if more precise positioning is desired. FIG. 4 also illustrates a line 404 along which the healthcare provider may cut the excess portion of the strap 202 that extends below the opening of the sensor connector 206. The healthcare provider may cut the excess portion below the locking mechanism, e.g., the cut may be higher in the orientation shown in FIG. 4, and may be within the opening of the sensor connector 206. The healthcare provider can discard the extra strap after the distance D has been set.

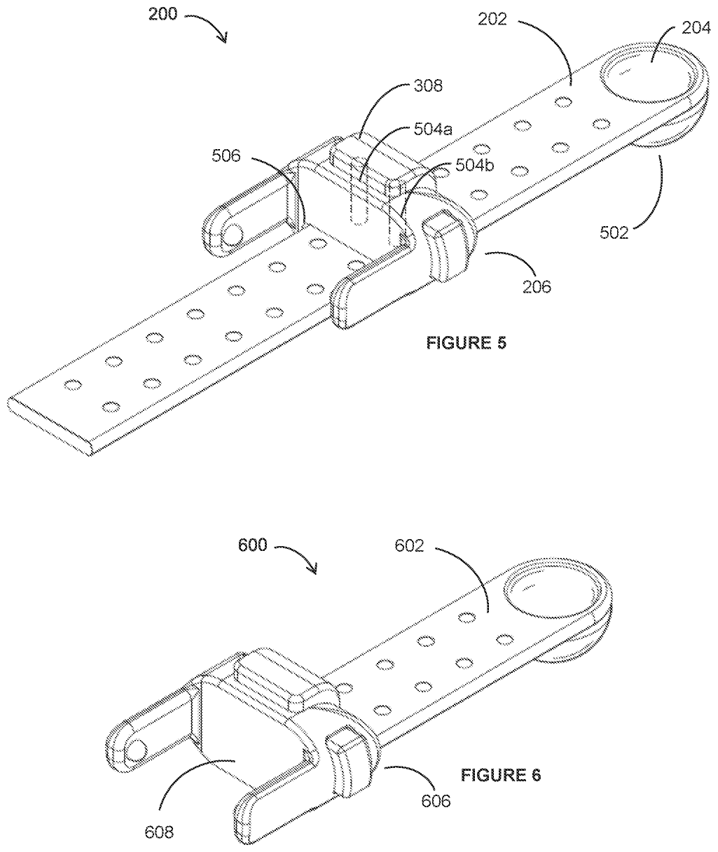

FIG. 5 illustrates a perspective view of the example alignment tool 200, according to some embodiments of the present disclosure. FIG. 5 shows a portion of the protruding region 502 that fits into a suprasternal notch. The protruding region 502 is rounded, e.g., the protruding region 502 may form a hemisphere or similar shape. The protruding region may be sized so that it fits within the suprasternal notch of most patients.

FIG. 5 also shows in outline the two pins 504a and 504b of the locking mechanism 308 described above. The pins 504 extend downward through the sensor connector 206 and through a corresponding pair of holes in the strap 202. FIG. 5 further shows the opening 506 in the sensor connector 206 that the strap 202 slides through.

In other embodiments, the locking mechanism 308 may lock the position of the strap 202 relative to the sensor connector 206 in other ways. For example, the locking mechanism 308 may include one or more of a clamp, a snap, a hook-and-loop closure, glue, or any other mechanism for permanently or non-permanently connecting the sensor connector 206 to a specific portion of the strap 202. If a different locking mechanism besides the pins 504 is used, the strap 202 may not include the holes 302.

### Alternate Alignment Tool Configurations

As shown in FIGS. 3-5, the strap 202 slides through a two-sided opening 506 that extends vertically through the sensor connector 206. As an alternative embodiment, the sensor connector may include a one-sided opening, or a receptacle, for the strap. FIG. 6 illustrates a perspective view of a second example alignment tool with a receptacle for the strap, according to some embodiments of the present disclosure.

In FIG. 6, the strap 602 is cut prior to being inserted into the receptacle. The strap 602 is cut based on a selected distance between the suprasternal notch and the sensor connector 606, in a similar manner to the length selection described above. The sensor connector 606 may include a locking mechanism similar to the locking mechanism 308 with pins described above, or another mechanism for engaging the strap 602. For example, in this example, a glue may be inserted into the receptacle and/or on the cut end of the strap 602 to hold the strap 602 in the receptacle. In this example, the lower face 608 of the sensor connector 606 is smooth, rather than including the opening 506 shown in FIG. 5. This may result in a more comfortable experience for the patient.

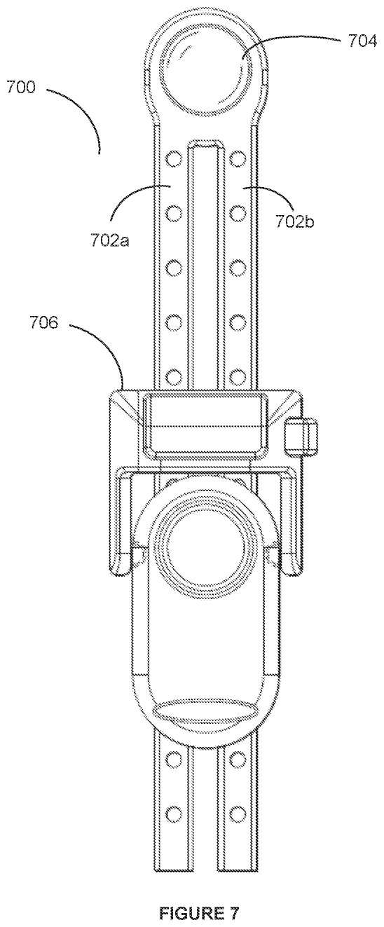

FIG. 7 illustrates a top view of a third example alignment tool, according to some embodiments of the present disclosure. In this example, rather than a strap 202, the alignment tool 700 has two arms 702a and 702b that extend from the suprasternal notch indent 704 and engage with the sensor connector 706. The sensor connector 706 may operate to selectively engage the UI housing in a similar manner to the sensor connector 206 described above. The suprasternal notch indent 704 is similar to the suprasternal notch indent 704 described with respect to FIGS. 2-5.

While two arms 702a and 702b are shown in FIG. 7, in other embodiments, the alignment tool 700 may have a single arm 702 or three or more arms 702. The arms 702 may be made of a rigid material, such as a rigid plastic. The sensor connector 706 may have a single opening, such as the opening 506, through which the arms 702 can be inserted. Alternatively, the sensor connector 706 may have multiple smaller openings, e.g., one for each of the arms 702. Alternatively, the sensor connector 706 may have a single receptacle or multiple receptacles (e.g., one for each arm), similar to the strap receptacle described with respect to FIG. 6.

In some embodiments, the sensor connector 706 includes a locking mechanism similar to the locking mechanism 308 described above. The locking mechanism may include pins that are inserted through corresponding holes in the arms 702. Alternatively, as described with respect to FIG. 5, the locking mechanism may include one or more of a clamp, a snap, a hook-and-loop closure, glue, or another mechanism for permanently or non-permanently connecting the sensor connector 706 to the arm or arms 702. If another locking mechanism besides pins is used, the arms 702 may not include holes.

### Select Examples

Example 1 provides an alignment tool that includes a sensor connector and a strap. The sensor connector engages with a sensor, e.g., a sensor device for monitoring cardiopulmonary conditions. The strap includes a first portion with a protruding region sized to fit into a suprasternal notch of a user and a second portion to engage with the sensor connector. The sensor connector and the first portion of the strap are separated by a distance having a configurable length.

Example 2 provides the alignment tool according to example 1, where the first portion of the strap includes a hollow portion corresponding to the protruding region on an opposite side of the strap from the protruding region.

Example 3 provides the alignment tool according to example 1 or 2, where the sensor connector includes an insertable pin for engaging a corresponding receptacle on the sensor.

Example 4 provides the alignment tool according to example 3, where the sensor connector further includes a button to retract the insertable pin of the sensor connector and disengage the sensor connector from the sensor.

Example 5 provides the alignment tool according to example 3 or 4, where the insertable pin is actuable by a single hand of the user.

Example 6 provides the alignment tool according to any of the preceding examples, where the second portion of the strap includes a series of holes positioned lengthwise along the strap, and the sensor connector includes a pin for insertion into a selected one of the holes to set the distance between the sensor connector and the first portion of the strap.

Example 7 provides the alignment tool according to example 6, where the pin is fixedly inserted into the selected hole such that a user cannot adjust the distance between the sensor connector and the first portion of the strap after the distance is set.

Example 8 provide the alignment tool according to example 6 or 7, where the series of holes is a first series of holes, the strap further includes a second series of holes positioned lengthwise along the strap, and the sensor connector further includes a second pin for insertion into a selected one of the second series of holes.

Example 9 provides the alignment tool according to example 8, where consecutive holes in the first series of holes and consecutive holes in the second series of holes are separated by approximately 1 centimeter.

Example 10 provides the alignment tool according to any of the preceding examples, where the sensor connector includes a receptacle for inserting the second portion of the strap, the second portion of the strap cut according to a selected distance between the first portion and the sensor connector.

Example 11 provides the alignment tool according to any of examples 1 through 9, where the sensor connector includes an opening for the second portion of the strap to slide through, the second portion of the strap slides within the opening to a position according to a selected distance between the first portion and the sensor connector.

Example 12 provides an alignment tool that includes a sensor connector to engage with a sensor, a suprasternal notch indent to fit into a suprasternal notch of a user, and at least one arm coupled to the sensor connector and coupled to the suprasternal notch indent, the arm providing a configurable length between the sensor connector and the suprasternal notch indent.

Example 13 provides the alignment tool according to example 12, where the suprasternal notch indent includes a hollow portion on one side and a corresponding protruding region on an opposite side, the protruding region sized to fit into the suprasternal notch of the user.

Example 14 provides the alignment tool according to example 12 or 13, where the sensor connector includes an insertable pin for engaging a corresponding receptacle on the sensor.

Example 15 provides the alignment tool according to example 14, where the sensor connector further includes a button to retract the insertable pin of the sensor connector and disengage the sensor connector from the sensor.

Example 16 provides the alignment tool according to any of examples 12 through 15, where the sensor connector includes a clamp to set a selected length between the sensor connector and the suprasternal notch indent.

Example 17 provides the alignment tool according to any of examples 12 through 16, where the at least one arm includes at least one series of holes positioned lengthwise along the arm, and the sensor connector includes at least one pin for insertion into a selected one of the holes to set a selected length between the sensor connector and the suprasternal notch indent.

Example 18 provides the alignment tool according to any of examples 12 through 17, where the sensor connector includes at least one opening for the at least one arm to slide through, the arm slides through the opening to a position according to a selected length between the sensor connector and the suprasternal notch indent.

Example 19 provides a sensor system that includes a chest sensor having a housing, the chest sensor to adhere to a chest of a user; and an alignment tool that includes a sensor connector to engage with the housing of the chest sensor and a strap that includes a first portion having a protruding region sized to fit into a suprasternal notch of a user, and a second portion to engage with the sensor connector, the first portion and the sensor connector separated by a distance having a configurable length.

Example 20 provides the sensor system according to example 19, where the chest sensor includes at least one receptacle, and the sensor connector includes at least one retractable pin for engaging the at least one receptacle of the chest sensor.

### Other Implementation Notes, Variations, and Applications

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

In one example embodiment, any number of electrical circuits of the figures may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), computer-readable non-transitory memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In various embodiments, the functionalities described herein may be implemented in emulation form as software or firmware running within one or more configurable (e.g., programmable) elements arranged in a structure that supports these functions. The software or firmware providing the emulation may be provided on non-transitory computer-readable storage medium comprising instructions to allow a processor to carry out those functionalities.

It is also imperative to note that all of the specifications, dimensions, and relationships outlined herein (e.g., the number of processors, logic operations, etc.) have only been offered for purposes of example and teaching only. Such information may be varied considerably without departing from the spirit of the present disclosure, or the scope of the appended claims. The specifications apply only to one non-limiting example and, accordingly, they should be construed as such. In the foregoing description, example embodiments have been described with reference to particular arrangements of components. Various modifications and changes may be made to such embodiments without departing from the scope of the appended claims. The description and drawings are, accordingly, to be regarded in an illustrative rather than in a restrictive sense.

Note that with the numerous examples provided herein, interaction may be described in terms of two, three, four, or more components. However, this has been done for purposes of clarity and example only. It should be appreciated that the system can be consolidated in any suitable manner. Along similar design alternatives, any of the illustrated components, modules, and elements of the FIGS. may be combined in various possible configurations, all of which are clearly within the broad scope of this Specification.

Note that in this Specification, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. Note that all optional features of the systems and methods described above may also be implemented with respect to the methods or systems described herein and specifics in the examples may be used anywhere in one or more embodiments.

In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke paragraph (f) of 35 U.S.C. Section 112 as it exists on the date of the filing hereof unless the words “means for” or “step for” are specifically used in the particular claims; and (b) does not intend, by any statement in the Specification, to limit this disclosure in any way that is not otherwise reflected in the appended claims.

What is claimed is:

1. An alignment tool comprising:
a sensor connector to engage with a sensor; and
a strap comprising:
a first portion comprising a protruding region sized to fit into a suprasternal notch of a user; and
a second portion to engage with the sensor connector;
the sensor connector and the first portion of the strap separated by a distance having a configurable length.

2. The alignment tool of claim 1, the first portion of the strap comprising a hollow portion corresponding to the protruding region on an opposite side of the strap from the protruding region.

3. The alignment tool of claim 1, the sensor connector comprising an insertable pin for engaging a corresponding receptacle on the sensor.

4. The alignment tool of claim 3, the sensor connector further comprising a button to retract the insertable pin of the sensor connector and disengage the sensor connector from the sensor.

5. The alignment tool of claim 3, the insertable pin actuable by a single hand of the user.

6. The alignment tool of claim 1, the second portion of the strap comprising a series of holes positioned lengthwise along the strap, and the sensor connector comprising a pin for insertion into a selected one of the holes to set the distance between the sensor connector and the first portion of the strap.

7. The alignment tool of claim 6, wherein the pin is fixedly inserted into the selected hole such that a user cannot adjust the distance between the sensor connector and the first portion of the strap after the distance is set.

8. The alignment tool of claim 6, wherein the series of holes is a first series of holes, the strap further comprising a second series of holes positioned lengthwise along the strap, and the sensor connector further comprising a second pin for insertion into a selected one of the second series of holes.

9. The alignment tool of claim 8, wherein consecutive holes in the first series of holes and consecutive holes in the second series of holes are separated by approximately 1 centimeter.

10. The alignment tool of claim 1, the sensor connector comprising a receptacle for inserting the second portion of the strap, the second portion of the strap cut according to a selected distance between the first portion and the sensor connector.

11. The alignment tool of claim 1, the sensor connector comprising an opening for the second portion of the strap to slide through, the second portion of the strap slides within the opening to a position according to a selected distance between the first portion and the sensor connector.

12. An alignment tool comprising:
a sensor connector to engage with a sensor;
a suprasternal notch indent to fit into a suprasternal notch of a user; and
at least one arm coupled to the sensor connector and coupled to the suprasternal notch indent, the arm providing a configurable length between the sensor connector and the suprasternal notch indent.

13. The alignment tool of claim 12, the suprasternal notch indent comprising a hollow portion on one side and a corresponding protruding region on an opposite side, the protruding region sized to fit into the suprasternal notch of the user.

14. The alignment tool of claim 12, the sensor connector comprising an insertable pin for engaging a corresponding receptacle on the sensor.

15. The alignment tool of claim 14, the sensor connector further comprising a button to retract the insertable pin of the sensor connector and disengage the sensor connector from the sensor.

16. The alignment tool of claim 12, the sensor connector comprising a clamp to set a selected length between the sensor connector and the suprasternal notch indent.

17. The alignment tool of claim 12, wherein the at least one arm comprises at least one series of holes positioned lengthwise along the arm, and the sensor connector comprises at least one pin for insertion into a selected one of the holes to set a selected length between the sensor connector and the suprasternal notch indent.

18. The alignment tool of claim 12, the sensor connector comprising at least one opening for the at least one arm to slide through, the arm slides through the opening to a position according to a selected length between the sensor connector and the suprasternal notch indent.

19. A sensor system comprising:
a chest sensor comprising a housing, the chest sensor to adhere to a chest of a user; and
an alignment tool comprising:
a sensor connector to engage with the housing of the chest sensor; and
a strap comprising:
a first portion comprising a protruding region sized to fit into a suprasternal notch of a user; and
a second portion to engage with the sensor connector;
the first portion and the sensor connector separated by a distance having a configurable length.

20. The sensor system of claim 19, the chest sensor comprising at least one receptacle, and the sensor connector comprising at least one retractable pin for engaging the at least one receptacle of the chest sensor.
